(19) United States
(12) Plant Patent Application Publication
(10) Pub. No.: US 2002/0152519 P1
(43) Pub. Date: Oct. 17, 2002

(54) Apple variety named 'Scifresh'

(76) Inventor: Allan G. White, Havelock North (NZ)

Correspondence Address: GREENLEE WINNER AND SULLIVAN P C, 5370 MANHATTAN CIRCLE, SUITE 201, BOULDER, CO, 80303, US

(21) Appl. No.: 09/834,757

(22) Filed: Apr. 13, 2001

(51) Int. Cl. A01H 5/00

(52) U.S. Cl. PLT/161

(57) ABSTRACT

A new and distinct apple variety is described. The variety results from selection among a population of seedlings derived from crossing the apple varieties known as ‘Braeburn’ (not patented) and ‘Royal Gala’ (U.S. Plant Pat. No. 4,121). The fruit of the apple tree of this new variety has an attractive appearance characterised by its striped colour pattern and is notable for its excellent eating quality post-storage. The new variety matures for harvest between the parent varieties and has been named ‘Scifresh’.

### BACKGROUND OF THE INVENTION

The invention described herein is a new variety of apple tree (Malus domestica). The new variety was selected from a population of seedlings derived from crossing the apple varieties ‘Braeburn’ and ‘Royal Gala’. The cross was made in 1985 on trees at Goddard Lane, Havelock North, Hawkes Bay, New Zealand. The new variety was determined to be distinct from the parent varieties ‘Braeburn’ and ‘Royal Gala’, as well as the sibling varieties ‘Scigold’ (not patented) and ‘Sweetie’ (not patented), by the following characteristics:

‘Braeburn’: ‘Scifresh’ has an earlier harvest season by at least a week, smaller fruit size, and the fruit shape is globose rather than conical, while fruit colour is a lighter red.

‘Royal Gala’: ‘Scifresh’ has a later harvest season, firmer flesh, and longer storage life.

‘Scigold’: ‘Scifresh’ fruit colour is red striped (whereas ‘Scigold’ is yellow-green), and has firmer flesh.

‘Sweetie’: ‘Scifresh’ has a later harvest season, more globose shape, and smaller fruit size.

The new variety has been named ‘Scifresh’. Asexual propagation by budding shows that the unique combination of characteristics of the variety come true to form and are established and transmitted through succeeding propagation. In order to obtain true-to-type clones of the initial plant, the breeders asexually propagated plants by budding dormant buds from the original seedling onto MM106 rootstock. The plants were asexually reproduced at Goddard Lane, Havelock North, Hawkes Bay, New Zealand.

### SUMMARY OF THE INVENTION

The new variety produces fruit with an attractive striped colour pattern and is notable for its excellent eating quality post-storage.

### BRIEF DESCRIPTION OF DRAWINGS

The accompanying photographs show typical specimens of the tree, foliage and fruit of the new variety as depicted in colours as nearly true as is reasonably possible to make the same in a colour illustration of this character.

FIG. 1: side view of a typical fruit of the apple variety ‘Scifresh’.

FIG. 2: calyx end view of a typical fruit of the apple variety ‘Scifresh’.

FIG. 3: stem end view of a typical fruit of the apple variety ‘Scifresh’.

FIG. 4: cross sectional view of a typical fruit of the apple variety ‘Scifresh’.

FIG. 5: two typical mature leaves of the variety ‘Scifresh’ showing the upper and lower surfaces.

FIG. 6: tree of the variety ‘Scifresh’ showing canopy and crop development prior to harvest.

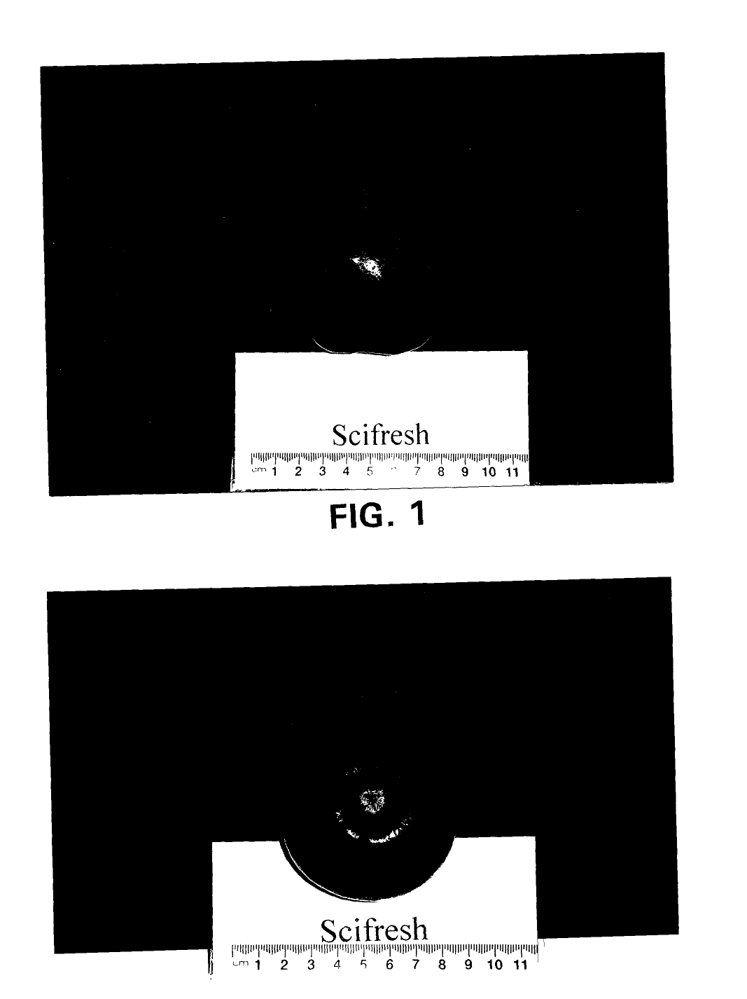

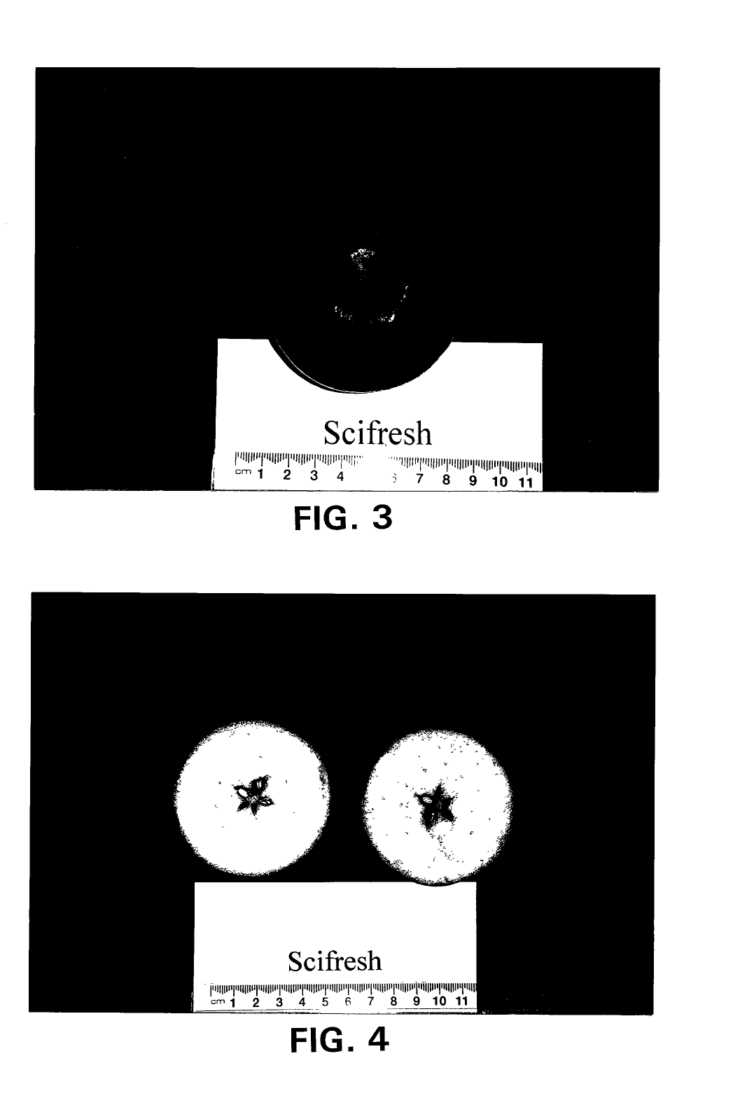

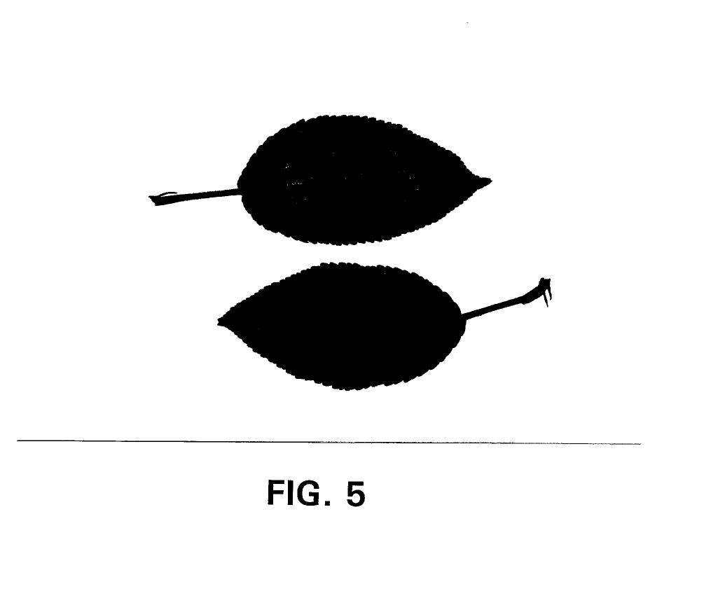

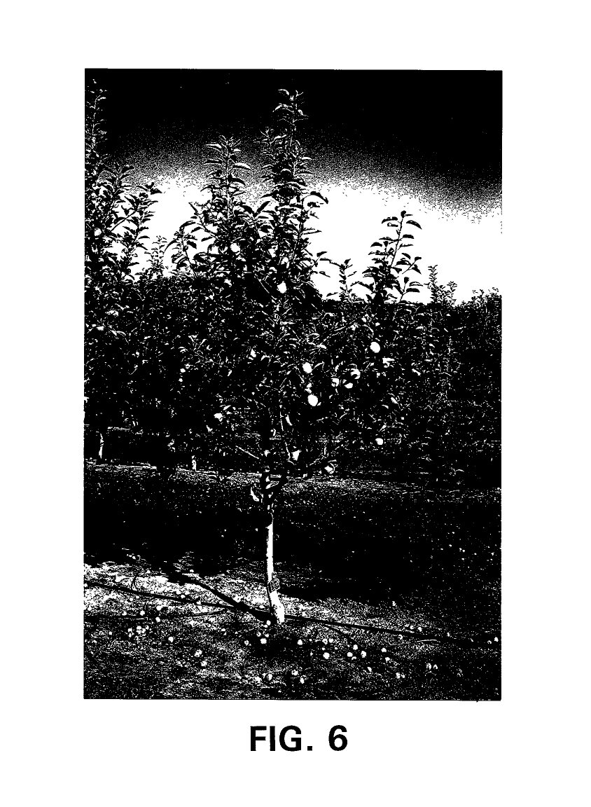

### DETAILED DESCRIPTION

The following is a detailed description of the new variety with colour terminology in accordance with The Royal Horticultural Society Colour Charts (R.H.S.C.C.) 1986 edition.

The specimens described were grown at Havelock North, Hawkes Bay, New Zealand. The observations were made in the 2000-2001 season on trees on MM. 106 rootstock that were three years old at the time, and managed under standard orchard practice.

Tree: Medium size; spreading habit; bearing on spurs; medium vigour on MM.106 rootstock.

Trunk: Moderately rough; diameter 45 mm at a height of 200 mm; the bark is grey-green R.H.S.S. 197C when mature.

Branches: Thin; moderately rough; moderate branching, the angle of branching being typically 20 degrees above the horizontal; approximately 6 spurs per 100 mm of growth on two year old wood; length of annual growth 300-400 mm.

Lenticels: Medium size; average numbers on current years growth.

Leaves: Length 91 mm; width 50 mm; upward pose; upfolded to concave shape in cross section; serrate indentations of margin; medium glossiness on upper surface; weak pubescence on lower side; petiole length 23-25 mm; medium stipule size; medium time of bud burst; colour of blade is yellow-green R.H.S.C.C. 147A.

Flower buds: Medium size, 12 mm long; red-purple R.H.S.C.C. 63B at popcorn stage.

Flowers: Medium blooming period, starting about October 5 for 2 weeks; five petals not touching; colour red R.H.S.C.C. 55B fading to white; five sepals medium size, pointed, green with red tips; many stamens with white filaments and yellow anthers; stigmatic surface typically at same level as anthers; pubescent ovary.

Fruit: Examined at optimum. Harvest period is from about March 12 until March 30 in Hawkes Bay, New Zealand.

Size: Small to medium; average width, 72 mm; average height, 65 mm; average weight, 177 g.

Shape: Ranges from short globose to conical; symmetrical in side view; ribbing absent; medium degree of crowning at distil end.

Cavity: Average width, 27 mm; average depth, 11 mm.

Basin: Average width, 23 mm; average depth, 9 mm.

Stem: Medium thickness; medium length, 22 mm.

Sepal: Medium length; spacing, touching.

Eye: Size, medium; aperture, closed.

Skin: Smooth; bloom of skin, absent; greasiness of skin, slight; cracking tendency of skin, absent; thickness, medium; background colour, yellow-green R.H.S.C.C. 154B; lenticels, small.

Over Colour: Approximately 75% of skin surface; red R.H.S.C.C. 179A striped; absent to low amount of russet around stem cavity.

Flesh: Firm, juicy and crisp; cream coloured R.H.S.C.C. 158A.

Texture: Medium.

Flavour: Medium sweetness; medium acidity; rich aroma.

Quality: Excellent.

Sinus: Closed.

Seeds: Five locules; 1 to 2 seeds per locule; medium seed size, 10 mm long, 5 mm wide; colour, brown R.H.S.C.C. 175A.

Use: Dessert.

Keeping quality: Excellent.

Production: Mid season, regular cropping.

Management: Thinning of crop load in early summer is recommended. Minimal pruning is required as the trees have a wide spreading habit.

We claim:

1. A new and distinct variety of apple plant, Malus domestica, substantially as herein shown and described.
